(19) United States
(12) Patent Application Publication
(10) Pub. No.: US 2025/0248745 A1
(43) Pub. Date: Aug. 7, 2025

(54) SPINAL ROD AND SPINAL FIXATION SYSTEM INCLUDING SAME

(71) Applicant: ORTHOPEDIATRICS CORP., Warsaw, IN (US)

(72) Inventors: Collin B. Gibbs, Columbia City, IN (US); Matthew Philip Prygoski, North Liberty, IN (US); Richard Detlefsen, Warsaw, IN (US); Camden L. Fox, Fort Wayne, IN (US); David W. Daniels, Winona Lake, IN (US)

(21) Appl. No.: 18/856,788

(22) PCT Filed: Apr. 14, 2023
(86) PCT No.: PCT/US2023/018718

§ 371 Date: Oct. 14, 2024

Related U.S. Application Data
(60) Provisional application No. 63/331,272, filed on Apr. 15, 2022.

(51) Int. Cl. A61B 17/70

(52) U.S. Cl. CPC A61B 17/7032; A61B 17/701; A61B 17/7076

(57) ABSTRACT

A spinal fixation system includes a bone anchor having a head, the head including a channel disposed therein and a channel axis. The channel includes an interior side surface and a lower surface. The spinal fixation system also includes a spinal rod having a textured surface configured to frictionally engage at least one of the interior side surface, the lower surface, a rod sleeve, or a set screw.

### CROSS REFERENCE TO RELATED APPLICATIONS

This application is a National Application No. PCT/US2023/018718, filed Apr. 14, 2023, which claims the priority benefit of U.S. Provisional Application No. 63/331,272, filed Apr. 15, 2022, each of which is incorporated herein by reference in its entirety.

### TECHNICAL FIELD

Embodiments of this technology relate in general to a rod implant and, more specifically, a spinal rod for use in spinal fixation systems.

### BACKGROUND

Spinal fixation systems may be used to surgically fix, adjust, and/or align the spinal column. One type of spinal fixation system employs a spinal rod for supporting the spine and fixing, adjusting, and/or aligning all or portions of the spinal column into a desired orientation. Attachment of the spinal rod to the spinal column has been achieved using a variety of vertebral anchors. Pedicle screws have been used successfully as vertebral anchors. Pedicle screws and connectors in combination with spinal rods can align and correct deformities in the natural spinal alignment as well as repair traumatic injury.

There remains an unmet need for an apparatus, system, and method to provide improved interaction between the spinal rod and a bone anchor.

### BRIEF DESCRIPTION OF THE DRAWINGS

The present disclosure will be more readily understood from a detailed description of some example embodiments taken in conjunction with the following figures:

FIG. 1 depicts a perspective view of one example of a spinal fixation system of the present disclosure including a pedicle screw and a spinal rod.

FIG. 2 depicts a side view of the spinal fixation system of FIG. 1 showing portions of the pedicle screw and spinal rod.

FIG. 3 depicts a perspective view of the pedicle screw head of the spinal fixation system of FIG. 1.

FIG. 4 depicts a side view of a portion of the spinal rod of the spinal fixation system of FIG. 1.

FIG. 5 depicts a plan view of the textured surface of the spinal rod of FIG. 4.

FIG. 6 depicts a side view of the textured surface of a portion of the spinal rod of FIG. 1.

FIG. 7 depicts a perspective view of the textured surface of a portion of the spinal rod of FIG. 1 highlighting a set of horizontal grooves.

FIG. 8 depicts a perspective view of the textured surface of a portion of the spinal rod of FIG. 1 highlighting crossed grooves.

FIG. 9 depicts a side view of one example of a rod sleeve of the spinal fixation system of FIG. 1.

FIG. 10 depicts a top perspective view of the rod sleeve of the spinal fixation system of FIG. 9.

FIG. 11 depicts a bottom perspective view of the rod sleeve of the spinal fixation system of FIG. 9.

FIG. 12 depicts a front view of a portion of one example of a spinal rod of the present disclosure.

FIG. 13 depicts a side cross-sectional view of one example of a spinal fixation system of the present disclosure.

FIG. 14 depicts a side cross-sectional view of one example of a set screw of the present disclosure.

FIG. 15 depicts a perspective view of one example of a spinal rod of the present disclosure.

FIG. 16 depicts a cross-sectional view of one example of a portion of a spinal fixation system of the present disclosure including a dimpled spinal rod.

FIG. 17 depicts a bottom perspective view of the set screw of the spinal fixation system of FIG. 16.

FIG. 18 depicts a front view of one example of a spinal rod of the present disclosure.

FIG. 19 depicts a cross-sectional view of one example of a spinal fixation system of the present disclosure including an externally-threaded set screw.

### DETAILED DESCRIPTION

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, it should be understood that unless otherwise specified or required by context, any explicit or implicit ordering of steps performed in the execution of a method does not imply that those steps must be performed in the order presented but instead may be performed in a different order or in parallel.

Described herein are example embodiments of a textured rod useful for such things as, for example, connecting and aligning bone anchors (e.g., hooks, screws, etc.) during orthopedic procedures such as, for example, spinal fixation. In the illustrated embodiments, a pedicle screw is disclosed to teach the features of a bone anchor and how the one or more embodiments of the textured rod work with such illustrative pedicle screws, but the disclosure is not to be limited only to pedicle screws, nor are the textured rods limited to engaging and/or working with only pedicle screws.

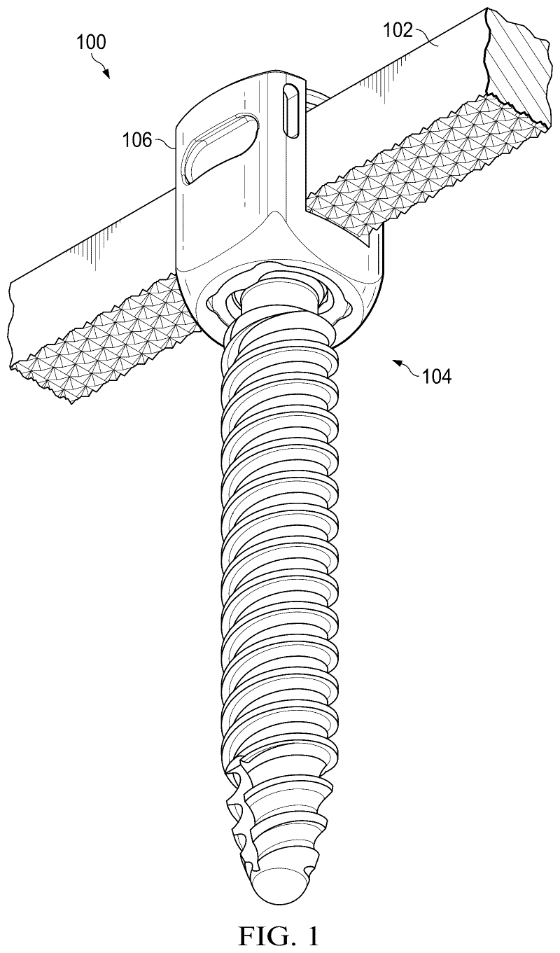

Referring to FIG. 1, a representative spinal fixation system 100 is shown. The spinal fixation system 100 includes a spinal rod 102 (e.g., a spinal fixation rod) and a bone anchor such as, for example, a pedicle screw 104, hooks, and other known bone anchors. The pedicle screw 104 can be any of known pedicle screws with any of known beneficial features for installation and use in processes and systems such as, for example, of spinal fixation. Example pedicle screws are described in International Patent Application Publication No. WO2020123857, which is incorporated herein by reference in its entirety. The pedicle screw 104 includes, for example, a screw shank, a neck, and a head 106. The head 106 can include first and second arms 108 opposed from each other. Each arm 108 of the head 106 includes a head pocket 110. The head pockets 110 are constructed to engage various orthopedic surgical tools configured to facilitate installation, removal, and/or adjustment of the pedicle screw 104 and/or one or more vertebrae that the pedicle screw is directly or indirectly connected thereto.

Referring to FIGS. 1-3, first and second arms 108 form a channel 112. First and second arms 108 can extend from a lower surface 114 of the interior channel 112, and each of the first and second arms may include an interior side surface. In an embodiment, the first and second arms 108 are at an angle of about 90°, such as from 85° to 95°, relative to the lower surface 114 of the interior channel 112. The channel 112 can be constructed to receive a spinal rod (e.g., spinal rod 102) and can include a channel axis. The channel axis can correspond to, and be coincident with, a longitudinal axis of the spinal rod 102 when it is reduced into the channel 112. A set screw 116 can be coupled to the head 106 to the desired position to secure and/or hold the spinal rod 102 in position within the channel 112 between the lower surface 114 of the channel 112 and the set screw 116. The proportions of the pedicle screw depicted are for illustrative purposes only and variations in the length, shape, and/or configuration of such pedicle screw may vary such as, for example, the shape and size of the head, the length of the shank, diameter of the screw, thread pitch, thread length, number of thread leads, shank induced compression and the like may be varied without departing from the scope of the disclosure.

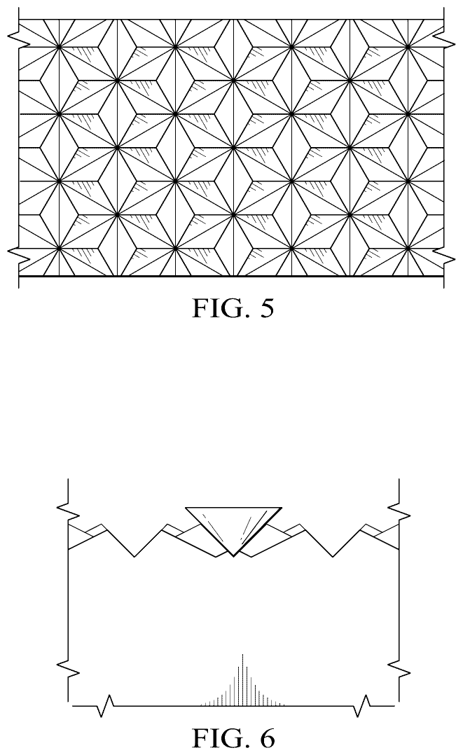

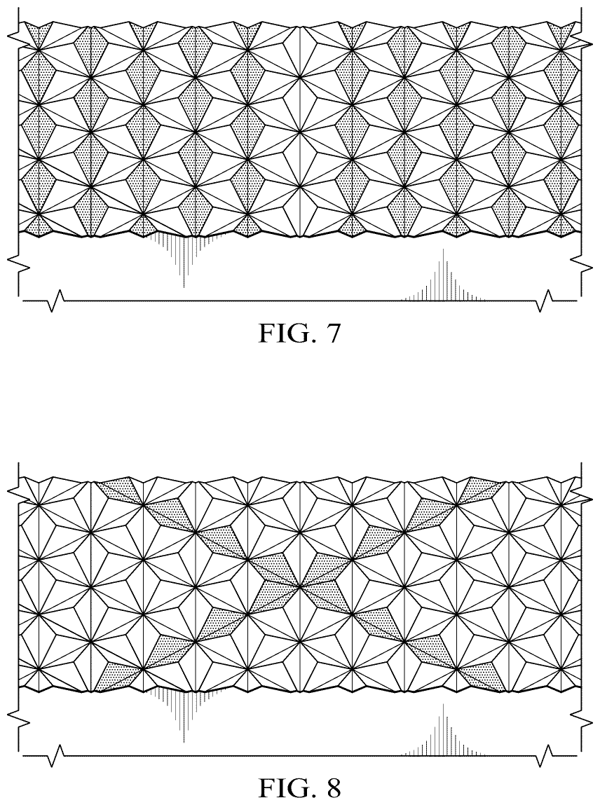

With reference to FIGS. 4-8, in various embodiments, the spinal rod 102 may have a textured surface. In the illustrated embodiment, a bottom surface 118 of the spinal rod 102 includes a grooved surface. The geometry of the grooved surface may be created by multiple passes of a cutter. For example, lines may be machined (e.g., milled) to create the geometry of the grooved surface. In a first example pass, a 90° cutter may be used to mill straight lines across the width of the spinal rod 102 (see highlighted lines in FIG. 7). The 90° cutter may be used to mill two straight lines that cross (e.g., in an X-pattern) in addition to the straight lines as shown in FIG. 8. The geometry or pattern of the grooved surface may vary. For example, the pattern may include only the first cut of straight lines highlighted in FIG. 7 or straight lines across the length (e.g., parallel to the longitudinal axis of the spinal rod 102. In another embodiment, the pattern may include only the X-shaped pattern highlighted in FIG. 8. The textured surface may, in various embodiments, extend along an entire length of the spinal rod 102 or may be segmented. For example, the spinal rod 102 may include alternating textured and smooth segments. The segments may be the same size or different sizes. For example, the alternative segments may be about 10 mm in length. The overall depth of the machined geometry could be, in an embodiment, in a range of 0.1 mm to 3.0 mm. A spinal rod 102, in an embodiment, may have areas with different patterns of the textured surface.

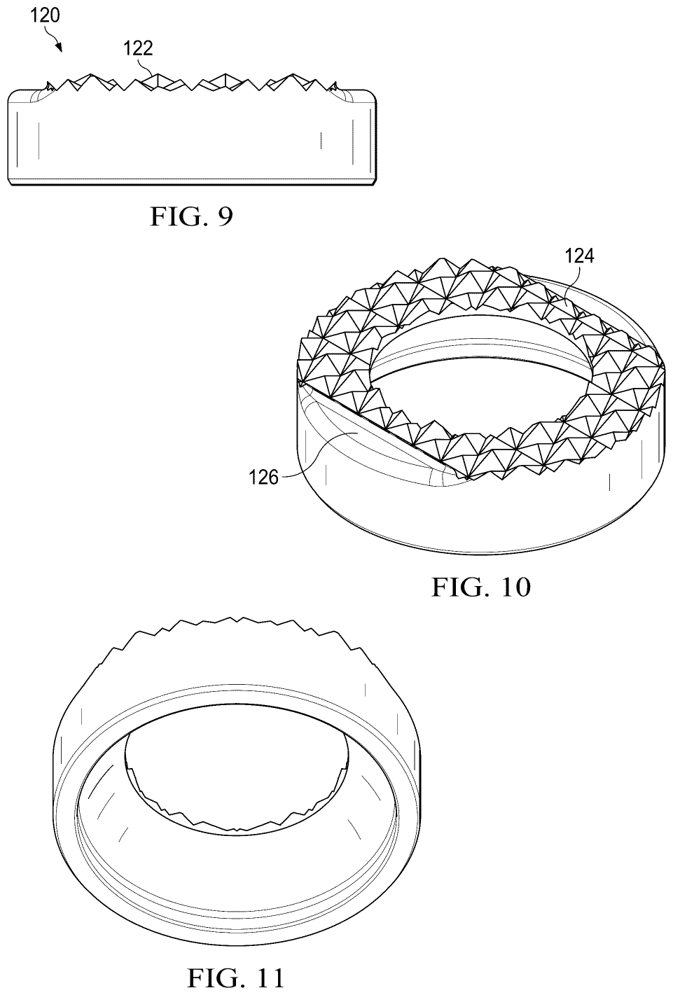

Referring to FIGS. 1 and 9-11, in various embodiments, the spinal fixation system 100 includes a rod sleeve 120. The rod sleeve 120 is configured to be positioned at the bottom of the channel 112. In an embodiment, the rod sleeve 120 sits on the lower surface 114 of the channel 112 between the arms 108. When inserted into the pedicle screw 104, the bottom surface 118 of the spinal rod 102 is in contact with a top surface 122 of the rod sleeve 120. In an embodiment, the rod sleeve 120 may have a standard machined finish. In some embodiments, the spinal rod 102 and the rod sleeve 120 may have a machined interference fit. For example, the rod sleeve 120 may include a textured surface. For example, a portion 124 or an entirety of the top surface 122 of the rod sleeve 120 has a pattern that corresponds to (e.g., a reciprocal of) the pattern on the bottom surface 118 of the spinal rod 102. The geometrical pattern on the bottom surface 118 of the spinal rod 102 and the top surface 122 of the rod sleeve 120 provide a machined interference fit between the surfaces. The interlocking nature provides increased grip strength between the spinal rod 102 and the rod sleeve 120. In an embodiment, the top surface 122 of the rod sleeve 120 includes two planar side portions 126 on opposing sides of the textured portion 124. In some embodiments, the textured portion 124 may have about the same width as the width of the spinal rod 102 and/or the width of the channel 112. The planar side portions 126 may extend out of the channel 112 and into the arms 108.

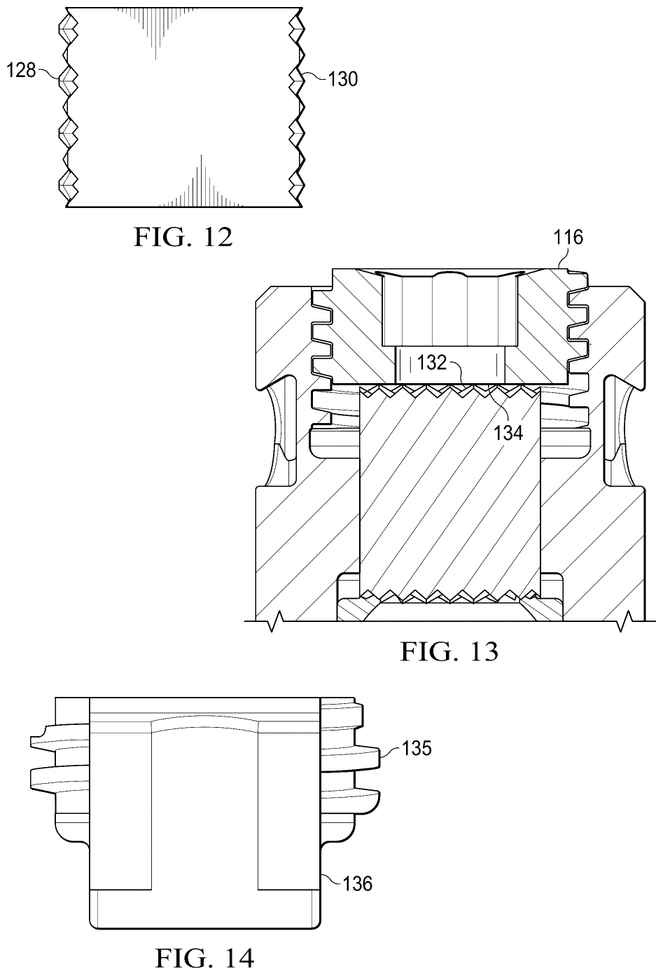

In embodiments where the spinal rod 102 includes a textured surface, the location of the textured surface may vary. For example, the textured surface may extend from one side to another side. The entire circumference of the spinal rod 102 may be textured. As shown in FIG. 12, in an embodiment, the spinal rod 102 may have opposing textured surfaces such as on the medial face 128 and lateral face 130. The inner surface of the arms 108 may have a corresponding texture to provide an interference fit between the medial and lateral faces 128, 130 of the spinal rod 102 and the arms 108. As another example, with reference to FIG. 13, the top surface 132 of the spinal rod 102 may have a textured surface in contact with the bottom surface 134 of the set screw 116. The set screw 116 could have either a textured or flat surface to mate with the top surface 132 of the spinal rod 102. An interference fit between the spinal rod 102 and a portion of the pedicle screw 104 may increase both the axial grip and rotational grip strengths of the system 100.

Referring to FIG. 14, in an embodiment, the set screw 116 may include a threaded set screw body 135 and a rotationally coupled bearing or pad 136. The rotatable bearing or pad 136 is configured to be in contact with the top surface 132 of the spinal rod 102. The bottom of the rotatable bearing or pad 136 may have a textured surface corresponding to the textured surface of the spinal rod 102 or may have a standard flat machined surface.

In embodiments where only one component of the spinal fixation system 100 includes a textured surface, the material of the component in contact with the textured surface may be made of a softer implant grade metal. For example, if the spinal rod 102 has a textured bottom surface 118, the top surface 122 of the rod sleeve 120 may be made of a softer metal than the textured bottom surface 118. The difference in hardness of the materials would allow for the non-textured surface to be “formed” around or imprinted by the opposing textured surface, which may provide increased axial grip strength and rotational grip strength.

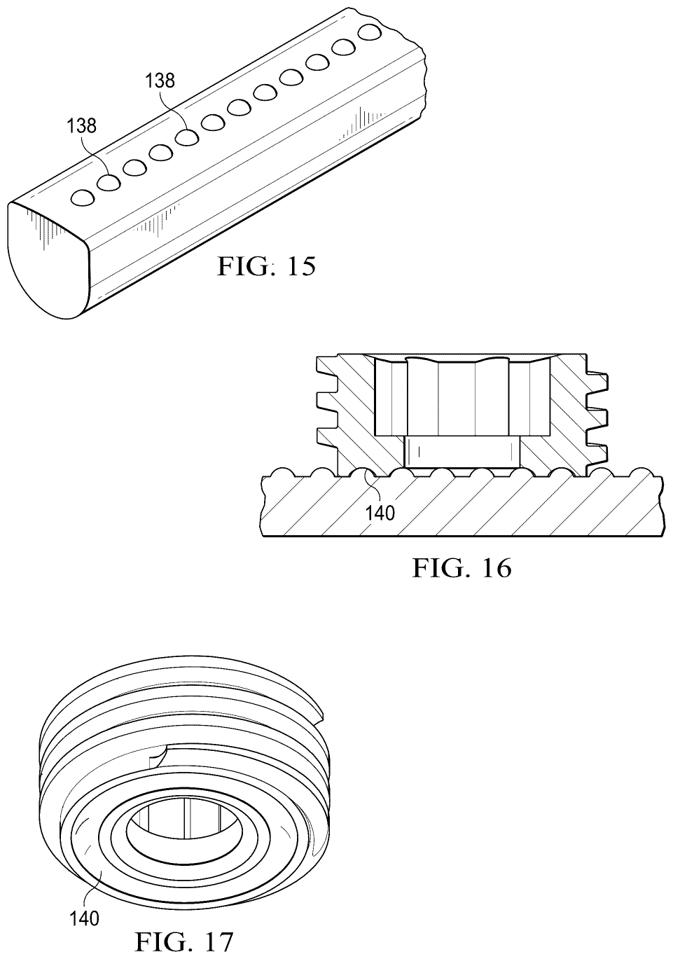

With reference to FIGS. 15-17, in an embodiment, the textured surface of the spinal rod 102 may be a dimpled surface. For example, the top surface 132 of the spinal rod 102 may be dimpled. In some embodiments, a portion of the pedicle screw 104 may be configured to capture one or more dimples 138 to provide increased grip strength. The set screw 116, in an embodiment, may have a bottom surface with one or more annular grooves 140. The annular grooves 140 may be sized to capture a dimple 138. The interference fit between the annular grooves 140 and dimples 138 may provide increase axial grip strength. The dimples 138 may be linearly aligned or staggered. The size and shape of the dimples 138 may vary. The shape of the dimples 138 can be circular, oblong, oval, rectangular, or a polygon shape with at least 2 parallel sides. The dimples 138 may have a height, for example, in a range of 0.1 mm to 3.0 mm. As described above, the dimpled textured surface may additionally or alternatively be present on any of the rod sleeve 120, the inner surfaces of the arms 108, or the set screw 116.

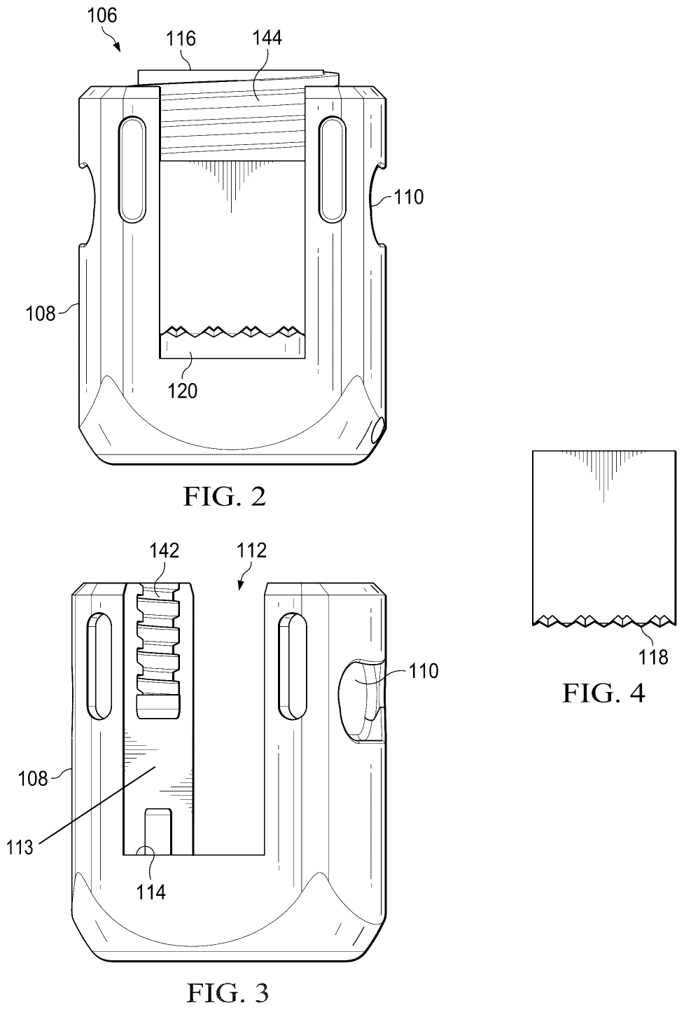

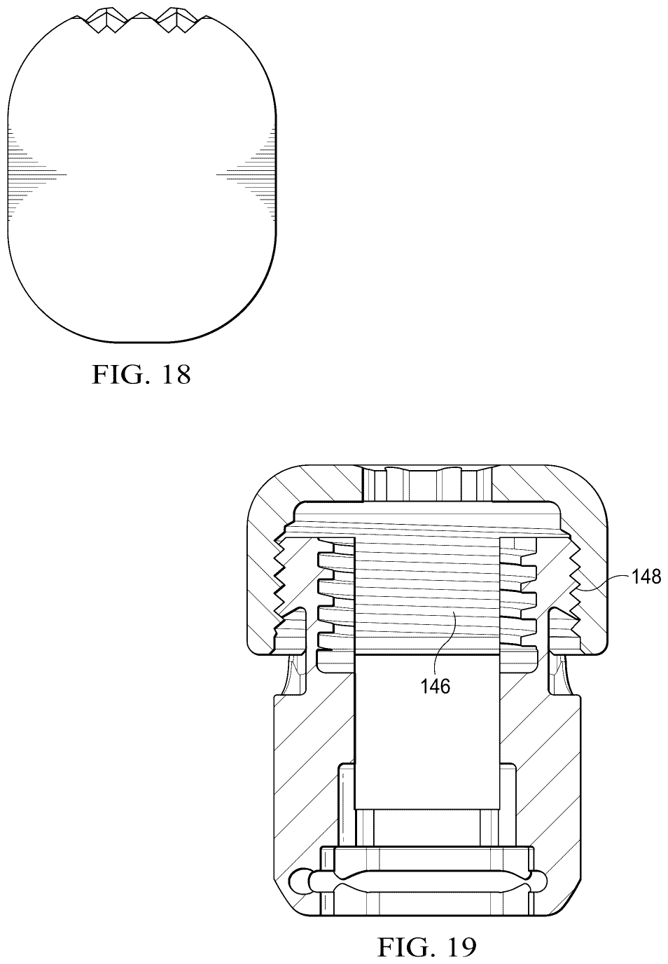

In an embodiment, the spinal rod 102 may have a circular cross section with a textured surface to provide increased axial grip strength. In various embodiments, the spinal rod 102 may have a non-circular cross section with or without a textured surface. Referring to FIGS. 2 and 12, the spinal rod 102 may have a rectangular cross section. As shown in FIG. 18, the spinal rod 102 may have an oval cross section. Other cross-sectional shapes may include a polygon with at least two parallel sides (e.g., square, hexagon, octagon, etc.). A spinal rod with a non-circular cross section may provide greater overall strength (e.g., bending, fatigue) compared to a circular spinal rod. In some embodiments, the spinal rod 102 may have both a non-circular cross section to provide increased rotational grip strength (i.e., to prevent rotation of the spinal rod 102 in the channel 112) and a textured surface to provide increased axial grip strength (i.e., to prevent longitudinal movement of the spinal rod 102 through the channel 112). Interlocking textured surfaces of the spinal rod 102 and pedicle screw 104 may also provide increased rotational grip strength.

The dimensions of the spinal rod 102 may vary and may be based on the intended application (e.g., size of the channel 112 in the pedicle screw). FIG. 4 shows example dimensions of 6.0 mm×7.5 mm. A non-rectangular rod may have the same footprint of 6.0 mm×7.5 mm. Another example rod footprint is 5.5 mm×5.5 mm.

Referring again to FIG. 3, in an embodiment, internal threads 142 are formed in arms 108. The thread profile of internal threads 142 may be any profile known to one skilled in the art. Non-limiting examples of internal threads 142 include reverse angle threads, square threads, ACME threads, and buttress threads. These internal threads 142 may be configured to receive and threadingly engage external threads 144 on the set screw 116.

With reference to FIG. 19, in an embodiment, internal threads 146 are formed in the set screw 116. The thread profile of internal threads 146 may be any profile known to one skilled in the art. Non-limiting examples of internal threads 146 include reverse angle threads, square threads, ACME threads, and buttress threads. These internal threads 146 may be configured to receive and threadingly engage external threads 148 on the arms 108. The internally-threaded set screw 116 constrains the exterior of the head 106 to further increase the axial grip strength and rotational grip strength.

In some embodiments, a kit includes one or more of a bone anchor (e.g., pedicle screw 104), a spinal rod (e.g., spinal rod 102), a set screw (e.g., set screw 116), and/or rod sleeve (e.g., rod sleeve 120).

In an embodiment, a method of use can include the following steps: inserting a pedicle screw having a head in a vertebral bone to a predetermined distance; placing a spinal rod in close proximity to, e.g., hovering over, the head of the inserted pedicle screw; seating the spinal rod into the head; and securing the spinal rod into the head.

A method of use for a spinal rod as described above can be described as a method for securing a spinal rod to a pedicle bone, the method including the following steps: providing a head of a pedicle screw; providing a spinal rod; seating the spinal rod into the head of the pedicle screw; and securing the spinal rod into the head of a pedicle screw.

In a first example, the spinal fixation system (100) includes a bone anchor (e.g., pedicle screw 104) that includes the head (106). The head includes the channel (112) disposed therein and a channel axis, and the channel including an interior side surface (113) and the lower surface (114); a spinal rod having a textured surface configured to frictionally engage at least one of the interior side surface, the lower surface, a rod sleeve, or a set screw.

In a second example, the textured surface of the spinal rod in the system of the first example is constructed to frictionally engage the lower surface. In a third example, the system of the second example may include the lower surface having an implant grade metal surface that is softer than the textured surface of the spinal rod such that the difference in the hardness between the textured surface of the spinal rod and the lower surface enables the lower surface to be deformed by the textured surface of the spinal rod. In a fourth example, the system of the first example may include the textured surface of the spinal rod disposed on at least one of a medial face or a lateral face of the spinal rod and constructed to engage the interior side surface. In a fifth example, the system of fourth example may include the head further including a first arm and a second arm that define the channel, at least one of the first arm and second arm include the interior side surface, and the interior side surface of the at least one of the first arm and second arm includes a textured surface constructed to engage the textured surface of the spinal rod.

In a sixth example, the system of the first example may further include a set screw including a bottom surface, wherein the textured surface of the spinal rod is constructed to engage the bottom surface of the set screw. In a seventh example, the bottom surface of the set screw of the system in the sixth example includes a textured surface constructed to engage the textured surface of the spinal rod. An eighth example system according to the seventh example, wherein the textured surface of the spinal rod is disposed on a top surface of the spinal rod.

A ninth example system according to the second example system above, wherein the spinal rod has a rectangular cross section. A tenth example system according to the ninth example system, wherein the textured surface of the spinal rod is disposed along the majority of a lower surface of the spinal rod. An eleventh example system according to the first example system above, wherein the bone anchor comprises a pedicle screw. A twelfth example system according to the first example system above, further comprising two or more bone anchors. Another example system according to the first example system above, further comprising two or more of the spinal rod.

An example method of using a spinal fixation system according to the first example system above, the method including: providing the head of the pedicle screw; providing the spinal rod; inserting a rod sleeve into the channel of the head; seating the spinal rod into the channel of the head; and securing the spinal rod into the head of the pedicle screw such that the textured surface of the spinal rod engages a surface of rod sleeve.

An example kit for a spinal fixation, the kit including: a bone anchor including a head, the head including a channel and a channel axis, the channel including an interior side surface and a lower surface; a spinal rod having a textured surface configured to frictionally engage at least one of the interior side surface, the lower surface, a rod sleeve, or a set screw. The example kit according to example kit above, further including a second bone anchor. One or more of the example kits of above, further including a second spinal rod. One or more of the example kits above, further including a rod sleeve. One or more of the example kits above, further comprising a set screw.

An example method of using a spinal fixation system, the method including: inserting a pedicle screw in a vertebral bone to a predetermined distance, the pedicle screw including a head and a channel; placing a rod sleeve into the channel, the rod sleeve including a top surface, wherein at least a portion of the top surface is a textured surface; placing a spinal rod in close proximity to the head of the inserted pedicle screw, the spinal rod including a textured surface disposed on a bottom surface of the spinal rod, the textured surfaces of the rod sleeve and spinal rod configured to engage each other; seating the spinal rod into the channel; and securing the spinal rod into the head such that the textured surfaces of the rod sleeve and spinal rod engage each other.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

What is claimed is:

1. A spinal fixation system (100) comprising:
a. a bone anchor (104) including a head (106), the head including a channel (112) disposed therein and a channel axis, and the channel including an interior side surface and a lower surface;
b. a spinal rod having a textured surface configured to frictionally engage at least one of the interior side surface, the lower surface, a rod sleeve, or a set screw.

2. The system according to claim 1, wherein the textured surface of the spinal rod is constructed to frictionally engage the lower surface.

3. The system according to claim 2, wherein the lower surface comprises an implant grade metal surface that is softer than the textured surface of the spinal rod such that the difference in the hardness between the textured surface of the spinal rod and the lower surface enables the lower surface to be deformed by the textured surface of the spinal rod.

4. The system according to claim 1, wherein the textured surface of the spinal rod is disposed on at least one of a medial face or a lateral face of the spinal rod and is constructed to engage the interior side surface.

5. The system according to claim 4, wherein the head further comprises a first arm and a second arm that define the channel, at least one of the first arm and second arm include the interior side surface, and the interior side surface of the at least one of the first arm and second arm includes a textured surface constructed to engage the textured surface of the spinal rod.

6. The system according to claim 1, further comprising a set screw including a bottom surface, wherein the textured surface of the spinal rod is constructed to engage the bottom surface of the set screw.

7. The system according to claim 6, wherein the bottom surface of the set screw includes a textured surface constructed to engage the textured surface of the spinal rod.

8. The system according to claim 7, wherein the textured surface of the spinal rod is disposed on a top surface of the spinal rod.

9. The system according to claim 2, wherein the spinal rod has a rectangular cross section.

10. The system according to claim 9, wherein the textured surface of the spinal rod is disposed along the majority of a lower surface of the spinal rod.

11. The system according to claim 1, wherein the bone anchor comprises a pedicle screw.

12. The system according to claim 1, further comprising two or more bone anchors.

13. The system according to claim 1, further comprising two or more of the spinal rod.

14. A method of using a spinal fixation system according to claim 1, the method comprising:
a. providing the head of the pedicle screw;
b. providing the spinal rod;
c. inserting a rod sleeve into the channel of the head;
d. seating the spinal rod into the channel of the head; and
e. securing the spinal rod into the head of the pedicle screw such that the textured surface of the spinal rod engages a surface of rod sleeve.

15. A kit for a spinal fixation, the kit comprising:
a. a bone anchor including a head, the head including a channel and a channel axis, the channel including an interior side surface and a lower surface;
b. a spinal rod having a textured surface configured to frictionally engage at least one of the interior side surface, the lower surface, a rod sleeve, or a set screw.

16. The kit according to claim 15, further comprising a second bone anchor.

17. The kit according to claim 15 further comprising a second spinal rod.

18. The kit according to claim 15 further comprising a rod sleeve.

19. The kit according to claim 15 further comprising a set screw.

20. A method of using a spinal fixation system, the method comprising:
a. inserting a pedicle screw in a vertebral bone to a predetermined distance, the pedicle screw including a head and a channel;
b. placing a rod sleeve into the channel, the rod sleeve including a top surface, wherein at least a portion of the top surface is a textured surface;
c. placing a spinal rod in close proximity to the head of the inserted pedicle screw, the spinal rod including a textured surface disposed on a bottom surface of the spinal rod, the textured surfaces of the rod sleeve and spinal rod configured to engage each other;
d. seating the spinal rod into the channel; and securing the spinal rod into the head such that the textured surfaces of the rod sleeve and spinal rod engage each other.
